(19) United States
(12) Patent Application Publication
(10) Pub. No.: US 2018/0064079 A1
(43) Pub. Date: Mar. 8, 2018

(54) REARING POD FOR REARING INSECTS

(71) Applicant: NORDIC INSECT ECONOMY LTD, Inkeroinen (FI)

(72) Inventors: Santeri HASA, Ikeroinen (FI); Jouni SAUKKONEN, Helsinki (FI); Sami VEKKELI, Tampere (FI); Santtu VEKKELI, Inkeroinen (FI); Arttu HIRVIKORPI, Turku (FI)

(21) Appl. No.: 15/255,448

(22) Filed: Sep. 2, 2016

(51) Int. Cl. A01K 67/033

(52) U.S. Cl. CPC A01K 67/033

(57) ABSTRACT

A rearing pod (100) for rearing insects includes a bottom part (110), a wall part (112), a rearing space (115) for insects, and an opening (117). The bottom and wall parts are foldable material. The bottom part is connected to a lower portion (113) of the wall part so that the bottom and wall parts define the space. An upper portion (114) of the wall part defines the opening and an inner surface (116) of the wall part faces the space. The upper portion includes a plastic strip (130) in order to prevent the insects to climb along the wall part. The strip is on the inner surface of the wall part and the strip covers continuously an inner surface (119) of the opening.

### TECHNICAL FIELD

The application relates generally to a rearing pod for rearing insects.

### BACKGROUND

Food-grade insects are traditionally reared in plywood or plastic rearing boxes, which are arranged parallelly in a rearing space.

In order to increase a rearing area of insects in the same space, it is used shelves in order to arrange the boxes parallelly on multiple layers.

### SUMMARY

One object of the invention is to withdraw the drawbacks of traditional rearing arrangements and provide a rearing pod, which is collapsible, lightweight, and easy to handle, and which allows to rear insects in multiple layers one pod on another.

One object of the invention is fulfilled by providing a rearing pod, a shelving frame, and a rearing system according to the independent claims.

One embodiment of the invention is a rearing pod for rearing insects that comprises a bottom part, a wall part, a rearing space for insects, and an opening. The bottom and wall parts are foldable material. The bottom part is connected to a lower portion of the wall part so that the bottom and wall parts define the space. An upper portion of the wall part defines the opening and an inner surface of the wall part faces the space. The upper portion comprises a plastic strip in order to prevent the insects to climb along the wall part. The strip is on the inner surface of the wall part and the strip covers continuously an inner surface of the opening.

One embodiment of the invention is a shelving frame for rearing pods that comprises vertical frame parts, connection frame parts, and horizontal frame parts. Each vertical frame part comprises a vertical front part, a vertical rear part, and at least two of the connection parts that connect the front and rear parts successively. The horizontal frame parts connect the vertical frame parts abreast in order to form the frame. Each vertical front and rear frame part comprises at least one rack part so that it is possible to hang up the rearing pods between the parallelly-connected vertical frame parts.

One embodiment of the invention is a rearing system for rearing insects that comprises a rearing pod and a shelving frame. The pod comprises a bottom part, a wall part, a rearing space for insects, and an opening. The bottom and wall parts are foldable material. The bottom part is connected to a lower portion of the wall part so that the bottom and wall parts define the space. An upper portion of the wall part defines the opening and an inner surface of the wall part faces the space. The upper portion comprises a plastic strip in order to prevent the insects to climb along the wall part. The strip is on the inner surface of the wall part and the strip covers continuously an inner surface of the opening. The shelving frame comprises vertical frame parts, connection frame parts, and horizontal frame parts. Each vertical frame part comprises a vertical front part, a vertical rear part, and at least two of the connection parts that connect the front and rear parts successively. The horizontal frame parts connect the vertical frame parts abreast in order to form the frame. Each vertical front and rear frame part comprises at least one rack part so that it is possible to hang up the rearing pods between the parallelly-connected vertical frame parts.

Further embodiments of the invention are defined in dependent claims.

### BRIEF DESCRIPTION OF THE FIGURES

The embodiments of the invention are presented with reference to the following figures:

FIG. 1 presents a rearing pod

FIG. 2 presents a shelving frame for rearing pods

FIG. 3 presents an attachment structure of frame

FIG. 4 presents how rearing pods are installed in a frame

### DETAILED DESCRIPTION OF THE FIGURES

FIG. 1 presents a rearing pod 100 for rearing food-grade insects on an industrial scale.

The insects can be e.g. maggots, e.g. mealworms, or crickets, or grasshoppers.

The pod 100 comprises a bottom part 110 and a wall part 112.

The bottom part 110 is connected to a lower portion 113 of the wall part 112 so that the parts 110, 112 form a rectangular bag (box)-like geometry of the pod 100 and define a rearing space 115 for insects.

The parts 110, 112 are manufactured from a semi-rigid foldable (collapsible) material, which keeps its intended form when it is properly supported. If the parts 110, 112 are without any support, it is possible to fold the parts 110, 112 into a very small volume in order to facilitate logistics.

In addition, the parts 110, 112 can be manufactured from a plastic, e.g. a polypropylene plastic.

Each of the parts 110, 112 can be manufactured from a single piece or several pieces.

The pod 100 can comprise the part 110, which is made from one piece, and the part 112, which is made from 2, 3, or 4 pieces.

In addition, the parts 110, 112 are arranged to surround the space 115 from five directions so that an inner surface of the part 110 and an inner surface 116 of the part 112 face the space 115.

The space 115 forms an incubation vessel for the insects. It is possible to increase a usable area of the space 115 by a rearing material.

The rearing material can be a honeycomb-like cardboard matrix or a cardboard chaff.

The space 115 makes possible to mount feed and water vessels inside the pod 100 without altering its geometry.

The part 112 comprises an upper portion 114 that defines an opening 117 and its size.

The opening 117 makes possible to put the insects and the rearing material into the space 115, to maintain the insects, and to remove necessary issues from the space 115 through the large opening 117.

The pod 100 can have following dimensions: a width w can be e.g. 900, 1000, or 1100 mm, a depth d can be e.g. 500, 600, or 700 mm, and a height h can be e.g. 500, 550, or 600 mm.

In addition, the pod 100 can comprise loop parts 120, which are arranged to an outer surface of the upper portion 114 or to an upper edge 118 of the upper portion 114, in order to attach at least one wooden suspension rod 122 to the pod 100.

The at least one rod 122 can comprise one rod, two rods according to the figure, or one rectangular or square rod frame.

The rod(s) 122 makes possible to move the pod 100 from one place to another and to hang up the pod 100 to a shelving frame 240.

In addition, the rod(s) 122 can rigidify the bag-like geometry.

In addition, the rod(s) 122 can be a bamboo rod(s).

The lower portion 113 can comprise at least two slots 124 for external support struts 126.

The slots 124 can comprise 2, 3, or 4 slots, and each slot 124 makes possible to install one wooden strut 126 inside it.

The struts 126, which are installed in the slots 124, rigidify the bag-like geometry.

In addition, the struts 126 can be bamboo struts.

However, the pod 100 retains sufficient geometry without the external support struts 126.

The upper portion 114 comprises a plastic strip 130 on the inner surface 116 so that it covers continuously an inner surface 119 of the opening 117.

The strip 130 can be manufactured from a single piece or several pieces.

In addition, the strip 130 can be manufactured from a polyethene plastic

A height s of the strip 130 can be e.g. 140, 150, or 160 mm.

In addition, the strip 130 can be attached by means of an attachment seam 132 so that an upper edge of the strip 130 is on the same level as the upper edge 118.

The seam 132 is attached e.g. 28, 30, 32 mm above a lower edge of the strip 130 so that it is formed a flap-like structure.

The slippery strip 130 prevents the insects to climb along the part 112 and escape from the pod 100.

In addition, the flap-like structure of the strip 130 prevents the insects to use the seam 132 in order to move along to seam 132 to inner corners 134 of the pod 100 and use the corners 134 in order to escape from the pod 100.

The strip 130 eliminates a need for a separate cover, whereupon it is easy to observe contents of the pods 100, and heat and gases can exchange freely.

The bag-like structure is lightweight, whereupon a single employee can handle it. In addition, the lightweight structure makes a cleaning and maintenance of the pod 100 easy, which improves hygiene in the pod 100 and its surroundings (a rearing space).

The pod 100 is easily emptied for separation of the contents due to its mobility and non-rigid structure when the supports 122, 126 have been removed.

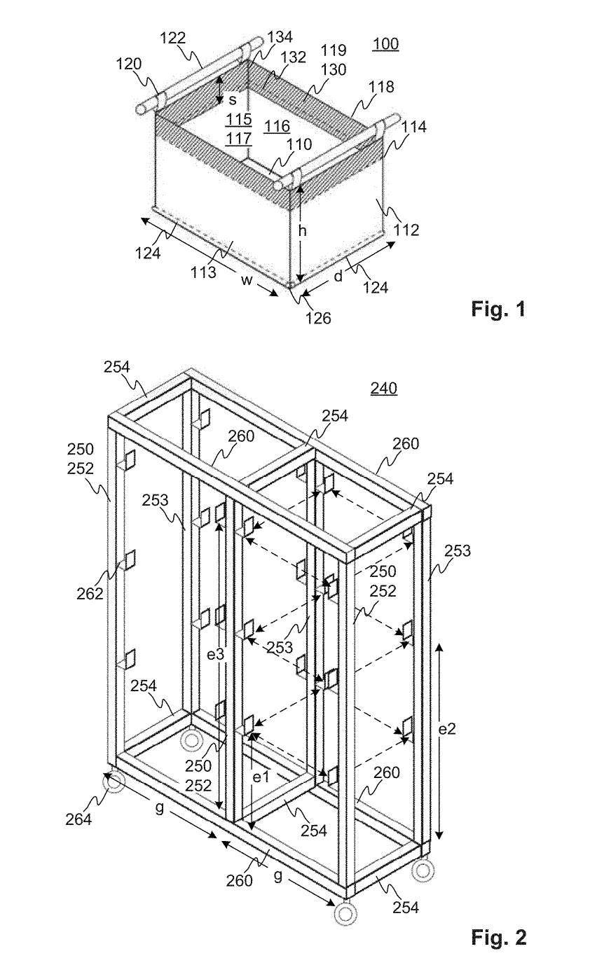

FIG. 2 presents the shelving frame 240 for rearing pods 110.

The frame 240 comprises vertical frame parts (walls) 250 and connection frame parts 254, and horizontal frame parts 260.

Each part 250 comprises a vertical front part 252, a vertical rear part 253, and at least two connection parts 254 that connect the front and rear parts 252, 253 successively so that part 250 has a rectangular shape.

In addition, the frame 240 comprises horizontal frame parts 260, which is used to connect the parts 250 abreast (parallelly) in order to form the frame 240.

The parts 250, 252, 253, 254, 260 can be wooden parts.

Each front and rear part 252, 253 comprises at least one attached rack part 262 so that it is possible to hang up at least one pod 100 between two parallelly-connected parts 250.

In addition, each front and rear part 252, 253 can comprise several attached suspension rack parts 262.

The rack parts 262 can be arranged between the parts 250 so that lowest four rack parts 262 are attached on an essentially same height (level) e1, centermost four rack parts 262 are attached on an essentially same height e2, and highest four rack parts 262 are attached on an essentially same height e3 according to the figure. The pods 100 can be hang up one on the other so that the lowest one is installed by means of the rods 122 on the rack parts 262 on the level e1, the centre-most on the rack parts 262 on the level e2, and the highest one on the rack parts 262 on the level e3.

The frame 240 can comprise three parallelly-connected parts 250 so that it is formed two gaps g between the parts 250 according to the figure. Such frame 240 makes possible to hang up three pods 100 one on the other into both gaps g.

In addition, the frame 240 can comprise wheels 264 in a bottom of the frame in order to move the frame.

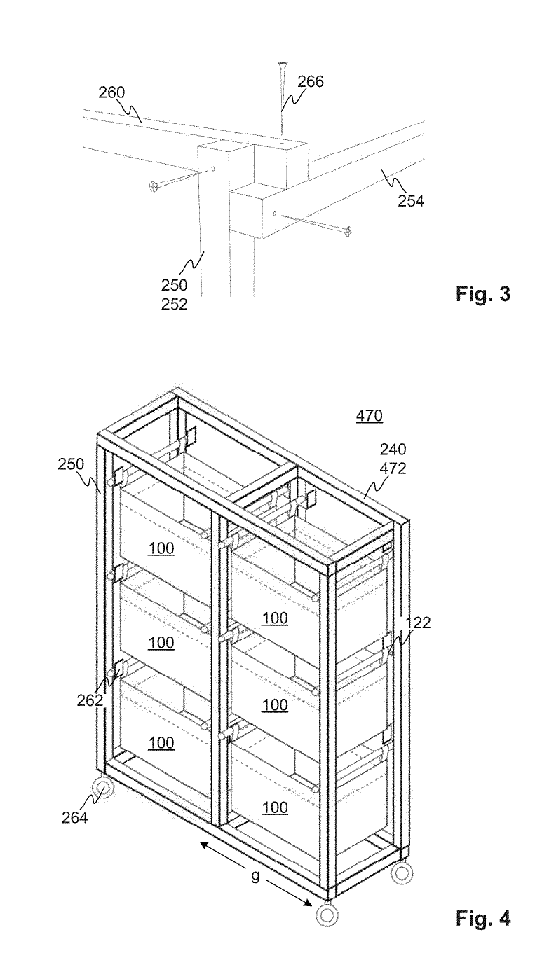

FIG. 3 presents how the connection parts 254 can be attached to the front and rear parts 252, 253 so that the parts 252, 253, 254 provide side-to-side interlocking joints in order to support the connected horizontal parts 260 an to rigidify a structure of the frame 240.

The parts 252, 253, 254, 260 are attached to each other by means of attaching means 266.

The means 266 can be nails or screws.

FIG. 4 presents a rearing system 470 for rearing insects, which comprises at least one pod 100 and the frame 240.

The rods 122 support the pods 100 and each supported pod 100 is placed in the frame 240 so that its rods 122 are mounted on the rack parts 262 according to the figure.

The rods 122 keeps the pods 100 in place, braces its structure to retain the bag-like geometry, and allows its rapid attachment and detachment.

The system 470 makes possible to mount several pods 100 on top of each other in several layers and allows a more efficient use of floor space in a rearing unit, which forms the rearing space. The frame 240 is fitted with the wheels 264, whereupon it is possible to move it in the rearing unit, which can comprise several frames 240 with the pods 100.

The system 470 can comprise a hydration system in order to provide a daily hydration of insects. The hydration can be done by addition of ca. 100 ml of tap-grade water into the water vessels of the pods 100.

The hydration system can comprise a dedicated piping assembly in a roof (472) of the system 470 and on the parts 250. The piping assembly comprises at least one water pipe in the roof 472.

In addition, the piping assembly comprises wall-mounted subassemblies, which diverge from the water pipe(s) installed in the roof 472 so that it is dedicated one subassembly to each gap g, whereupon it is possible to supply the pods 100 in each gap g.

Each subassembly comprises at least one water pipe, which is mounted on the part 250.

In addition, the piping assembly comprises final water distribution pipes, which diverge from the water pipe(s) of each subassembly in order to supply the individual pods 100.

Each final water distribution pipe comprises one water pipe, which is connected to the subassembly.

A mounting of the water pipes is rigid so that the water pipes accurately connect to the water vessels of the individual pods 100.

The water pipes in the piping assembly can be plastic and/or metal pipes.

In addition, the hydration system can comprise an automated and timer-equipped pumping unit, which executes a dosage and dispensing of water. The water requirement is approximate, which renders minor inaccuracies caused by pressure differentials of the pumping unit negligible.

The invention has been now explained above with reference to the aforementioned embodiments and the several advantages of the invention have been demonstrated.

It is clear that the invention is not only restricted to these embodiments, but comprises all possible embodiments within the scope of the following claims.

1. A rearing pod (100) for rearing insects comprising
a bottom part (110),
a wall part (112),
a rearing space (115) for insects, and
an opening (117),
wherein the bottom and wall parts are foldable material,
wherein the bottom part is connected to a lower portion (113) of the wall part so that the bottom and wall parts define the space,
wherein an upper portion (114) of the wall part defines the opening, and
wherein an inner surface (116) of the wall part faces the space,
wherein
the upper portion comprises a plastic strip (130) in order to prevent the insects to climb along the wall part,
wherein the strip is on the inner surface of the wall part and
wherein the strip covers continuously an inner surface (119) of the opening.

2. The pod of claim 1, which comprises loop parts (120) in the upper portion of the wall part in order to attach suspension rods (122) to the pod.

3. The pod of claim 1, wherein the lower portion of the wall part comprises at least two slots (124) for external support struts (126), which rigidify a bag-like geometry of the pod.

4. The pod of claim 1, wherein the bottom and wall parts are polypropylene plastic and the strip is polyethene plastic.

5. A shelving frame (240) for rearing pods (110) of claim 1 comprising
vertical frame parts (250),
connection frame parts (254), and
horizontal frame parts (260),
wherein each vertical frame part (250) comprises a vertical front part (252), a vertical rear part (253), and at least two of the connection parts that connect the front and rear parts (252, 253) successively, and
wherein the horizontal frame parts connect the vertical frame parts abreast in order to form the frame,
wherein
each vertical front and rear frame part (252, 253) comprises at least one rack part (262) so that it is possible to hang up the rearing pods between the parallelly-connected vertical frame parts.

6. The frame of claim 5, wherein rack parts (262) are attached to the vertical frame parts so that it is possible to hang up the rearing pods one on the other between the vertical frame parts.

7. The frame of claim 5, which comprises three parallelly-connected vertical frame parts (250) so that it is formed two gaps (g) between the three vertical frame parts and it is possible to hang up the rearing pods one on the other into the gaps.

8. The frame of claim 5, which comprises wheels (264) in a bottom of the frame in order to move the frame.

9. The frame of claim 5, wherein the vertical frame, connection, and horizontal frame parts are wooden parts, and the connection parts are attached to the vertical front and rear parts (252, 253) so that those support the connected horizontal frame parts.

10. A rearing system (470) for rearing insects comprising a rearing pod (100) according to claim 1 and a shelving frame (240) for a plurality of rearing pods, comprising vertical frame parts (250), connection frame parts (254), and horizontal frame parts (260),
wherein each vertical frame part (250) comprises a vertical front part (252), a vertical rear part (253), and at least two of the connection parts that connect the front and rear parts (252, 253) successively,
the horizontal frame parts connect the vertical frame parts abreast in order to form the frame, and
each vertical front and rear frame part (252, 253) comprises at least one rack part (262) so that it is possible to hang up the rearing pods between the parallelly-connected vertical frame parts.

11. The pod of claim 2, wherein the lower portion of the wall part comprises at least two slots (124) for external support struts (126), which rigidify a bag-like geometry of the pod.

12. The frame of claim 6, which comprises three parallelly-connected vertical frame parts (250) so that it is formed two gaps (g) between the three vertical frame parts and it is possible to hang up the rearing pods one on the other into the gaps.

13. The frame of claim 6, which comprises wheels (264) in a bottom of the frame in order to move the frame.

14. The frame of claim 7, which comprises wheels (264) in a bottom of the frame in order to move the frame.

15. The frame of claim 6, wherein the vertical frame, connection, and horizontal frame parts are wooden parts, and the connection parts are attached to the vertical front and rear parts (252, 253) so that those support the connected horizontal frame parts.

16. The frame of claim 7, wherein the vertical frame, connection, and horizontal frame parts are wooden parts, and the connection parts are attached to the vertical front and rear parts (252, 253) so that those support the connected horizontal frame parts.

17. The frame of claim 8, wherein the vertical frame, connection, and horizontal frame parts are wooden parts, and the connection parts are attached to the vertical front and rear parts (252, 253) so that those support the connected horizontal frame parts.
